(19) United States
(12) Patent Application Publication
(10) Pub. No.: US 2025/0340862 A1
(43) Pub. Date: Nov. 6, 2025

(54) Methods and Compositions for Obtaining Linked Image and Sequence Data for Single Cells

(71) Applicant: Becton, Dickinson and Company, Franklin Lakes, NJ (US)

(72) Inventors: Adam Thomas Wright, San Diego, CA (US); Hye Won Song, San Diego, CA (US); Jody Martin, Ashland, OR (US)

(21) Appl. No.: 18/854,255

(22) PCT Filed: Apr. 12, 2023
(86) PCT No.: PCT/US2023/018375

§ 371 Date: Oct. 4, 2024

Related U.S. Application Data
(60) Provisional application No. 63/332,087, filed on Apr. 18, 2022.

(51) Int. Cl. C12N 15/10; G01N 33/68

(52) U.S. Cl. CPC C12N 15/1065; C12N 15/1072; G01N 33/6845

(57) ABSTRACT

Aspects of the invention include methods of obtaining linked image and sequence data for single cells, e.g., of a cellular sample. Embodiments of the methods include: combinatorially barcoding cells, e.g., obtained from an initial cellular sample, with specific binding member/oligonucleotide sub-barcodes to produce combinatorial barcoded cells. The resultant combinatorial barcoded cells are next partitioned to produce partitioned combinatorial barcoded single cells each having a combinatorial barcode. Image data and sequence data are then obtained for the partitioned combinatorial barcoded single cells, followed by linking of the image data and sequence data that share a common combinatorial barcode in order to obtain linked image and sequence data for single cells of the cellular sample. Also provided are compositions for practicing methods of the invention.

### CROSS-REFERENCE TO RELATED APPLICATION

Pursuant to 35 U.S.C. § 119 (e), this application claims priority to the filing date of U.S. Provisional Patent Application Ser. No. 63/332,087 filed Apr. 18, 2022; the disclosure of which application is incorporated herein by reference in its entirety.

### INTRODUCTION

Current technology allows for measurement of gene expression of single cells in a massively parallel manner (e.g., >10,000 cells) by attaching cell specific oligonucleotide barcodes to poly(A) mRNA molecules from individual cells as each of the cells is co-localized with a barcoded reagent bead in a compartment. One platform that allows measurement of gene expression of single cells in a massively parallel manner is the BD Rhapsody™ Single-Cell Analysis System. The BD Rhapsody™ Single-Cell Analysis System is a platform that allows high-throughput capture of nucleic acids from single cells using a simple cartridge workflow and a multitier barcoding system. The resulting captured information can be used to generate various types of next-generation sequencing (NGS) libraries, including libraries suitable for whole transcriptome analysis, e.g., for discovery biology and targeted RNA analysis for high sensitivity transcript detection. Shum et al., “Quantitation of mRNA Transcripts and Proteins Using the BD Rhapsody™ Single-Cell Analysis System,” Adv Exp Med Biol. 2019; 1129:63-79.

Gene expression may affect protein expression. Protein-protein interaction may affect gene expression and protein expression. As such, more recently systems and methods that can quantitatively analyze protein expression in cells, and simultaneously measure protein expression and gene expression in cells, have been developed. One such platform the BD Abseq platform. AbSeq is a method to profile proteins in single cells. In Abseq, the usual fluorophore labeled antibodies are replaced with nucleic acid sequence tags that can be read out at the single-cell level, e.g., via barcoding and NGS sequencing. “The objective of Abseq is to enable the sensitive, accurate, and comprehensive characterization of proteins in large numbers of single cells. Cells are bound with antibodies against the different target epitopes, as in conventional immunostaining, except that the antibodies are labeled with unique sequence tags. When an antibody binds its target, the DNA tag is carried with it, allowing the presence of the target to be inferred based on the presence of the tag. In this way, counting tags provides an estimate of the different epitopes present in the cell, as detected via antibody binding.” Shahi et al., “Abseq: Ultrahigh-throughput single cell protein profiling with droplet microfluidic barcoding. Sci Rep 7, 44447 (2017).”

### SUMMARY

The inventors have realized that it would be desirable to link image data to massively parallel NGS data in single cell analysis, including single cell multiomic applications. The inventors are not aware of any current protocol that exists to link single cell imaging data to single cell multiomic data from the same cell. While one can first single cell sort (FACS) cells into macro-well plates (96-well, or other) and then perform plate based single cell multiomic workflows on the sorted cells, such does not provide image data linked to NGS data for the cells. Plate-based workflows do not offer the same throughput or efficiency as massively parallel single cell multiomic workflows. Furthermore, the indexed data is not microscope based and currently employed common flow cytometer data lacks 2 dimensional (spatial) information. Embodiments of the invention satisfy the need in the art for methods and compositions to readily obtain linked image and sequencing data for single cells.

Aspects of the invention include methods of obtaining linked image and sequence data for single cells, e.g., of a cellular sample. Embodiments of the methods include: combinatorially barcoding cells, e.g., obtained from an initial cellular sample, with specific binding member/oligonucleotide sub-barcodes to produce combinatorial barcoded cells. The resultant combinatorial barcoded cells are next partitioned to produce partitioned combinatorial barcoded single cells each having a combinatorial barcode. Image data and sequence data are then obtained for the partitioned combinatorial barcoded single cells, followed by linking of the image data and sequence data that share a common combinatorial barcode in order to obtain linked image and sequence data for single cells of the cellular sample. Also provided are compositions for practicing methods of the invention.

### BRIEF DESCRIPTION OF THE FIGURES

The invention may be best understood from the following detailed description when read in conjunction with the accompanying drawings. Included in the drawings are the following figures:

FIG. 1 schematically illustrates split-pool sample indexing down to individual cells, in accordance with an embodiment of the invention.

FIG. 2 schematically illustrates Sslit/Pool ab-oligo labeling of cells to generate single cell indices that can be read by imaging as well as downstream single cell multiomics, in accordance with an embodiment of the invention.

FIG. 3 provides an example of using cyclic Immuno-Fluorescence to decode ab-oligo signature of individual cells, in accordance with an embodiment of the invention.

### DEFINITIONS

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, an antibody can be a full-length (e.g., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. In some embodiments, an antibody is a functional antibody fragment. For example, an antibody fragment can be a portion of an antibody such as F(ab′)2, Fab′, Fab, Fv, sFv and the like. An antibody fragment can bind with the same antigen that is recognized by the full-length antibody. An antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (for example, CD8, CD34, and CD45), and therapeutic antibodies.

As used herein the term “associated” or “associated with” can mean that two or more species are identifiable as being co-located at a point in time. An association can mean that two or more species are or were within a similar container. An association can be an informatics association. For example, digital information regarding two or more species can be stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label. An association can comprise hybridization between two molecules (such as a target molecule and a label).

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (e.g., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one).

As used herein, the term “sample” can refer to a composition comprising targets. Suitable samples for analysis by the disclosed methods, devices, and systems include cells, tissues, organs, or organisms. A cellular sample is a composition that is made up of multiple cells, such as a composition that includes multiple disparate cells, such as an aqueous composition of single cells, where the number of cells may vary.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which nucleic acids may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A plurality of solid supports spaced in an array may not comprise a substrate. A solid support may be used interchangeably with the term “bead.”

As used here, the term “target” can refer to a composition which can be analyzed in accordance with embodiments of the invention. Exemplary suitable targets for analysis by the disclosed methods, devices, and systems include oligonucleotides, DNA, RNA, mRNA, microRNA, tRNA, and the like. Targets can be single or double stranded. In some embodiments, targets can be proteins, peptides, or polypeptides. In some embodiments, targets are lipids. As used herein, “target” can be used interchangeably with “species.”

As used herein, the term “reverse transcriptases” can refer to a group of enzymes having reverse transcriptase activity (i.e., that catalyze synthesis of DNA from a RNA template). In general, such enzymes include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants or derivatives thereof. Non-retroviral reverse transcriptases include non-LTR retrotransposon reverse transcriptases, retroplasmid reverse transcriptases, retron reverse transciptases, and group II intron reverse transcriptases. Examples of group II intron reverse transcriptases include the Lactococcus lactis Ll.LtrB intron reverse transcriptase, the Thermosynechococcus elongatus Tel4c intron reverse transcriptase, or the Geobacillus stearothermophilus Gsl-IIC intron reverse transcriptase. Other classes of reverse transcriptases can include many classes of non-retroviral reverse transcriptases (i.e., retrons, group II introns, and diversity-generating retroelements among others).

### DETAILED DESCRIPTION

Aspects of the invention include methods of obtaining linked image and sequence data for single cells, e.g., of a cellular sample. Embodiments of the methods include: combinatorially barcoding cells, e.g., obtained from an initial cellular sample, with specific binding member/oligonucleotide sub-barcodes to produce combinatorial barcoded cells. The resultant combinatorial barcoded cells are next partitioned to produce partitioned combinatorial barcoded single cells each having a combinatorial barcode. Image data and sequence data are then obtained for the partitioned combinatorial barcoded single cells, followed by linking of the image data and sequence data that share a common combinatorial barcode in order to obtain linked image and sequence data for single cells of the cellular sample. Also provided are compositions for practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### METHODS

As summarized above, methods of obtaining linked image and sequence data for single cells, e.g., of an initial cellular sample, are provided. By linked image and sequence data is meant a combined data set that includes both image data and nucleic acid sequence data that can be attributed to the same cell, such that it can be considered as originating from the same cell. In other words, linked image and sequence data is a data set the includes both image data and nucleic acid sequence data that is obtained from the same cell. Image data is data obtained from a cell using an imaging technique. The term “image” is used in its conventional sense to refer to a representation of an object, e.g., cell, produced by means of radiation, e.g., via illumination with light. Image data is data that collectively makes up the representation, and may be data obtained using any convenient protocol. In some embodiments, image data obtained in methods of the invention is microscopy image data. Microscopy image data refers to image data obtained using microscopes to view objects and areas of objects, e.g., cells, that cannot be seen with the naked eye. Nucleic acid sequence data refers to data obtained using a nucleic acid sequencing technique, which identifies the sequence of nucleotides in a nucleic acid molecules. Nucleic acid sequencing data from a cell includes the sequence of one or more nucleic acid sequences, e.g., RNA molecules, present in the cell. Such data may be obtained using a variety of sequence protocols, including next generation sequence (NGS) protocols.

As summarized above, aspects of the methods include: combinatorially barcoding cells of a cellular sample with specific binding member/oligonucleotide sub-barcodes to produce combinatorial barcoded cells; partitioning the combinatorial barcoded cells to produce partitioned combinatorial barcoded single cells each having a combinatorial barcode; obtaining image data and sequence data for the partitioned combinatorial barcoded single cells; and linking the image data and sequence data that share a common combinatorial barcode; to obtain linked image and sequence data for single cells of the cellular sample. Embodiments of each of these steps is now described in greater detail.

Combinatorially Barcoding Cells of a Cellular Sample with Specific Binding Member/Oligonucleotide Sub-Barcodes

Embodiments of the methods include combinatorially barcoding cells of a cellular sample with specific binding member/oligonucleotide sub-barcodes. By combinatorially barcoded cells is meant that cells of an initial cellular sample are modified to have stably associated therewith a unique combination of sub-barcodes (provided by a combination of specific binding member/oligonucleotide sub-barcodes), which unique combination collectively makes up a unique combinatorial barcode for that cell. By stably associated therewith is meant that the specific binding member/oligonucleotide sub-barcodes making up a given combinatorial barcoded of a combinatorially barcoded cell are attached to the surface of that cell in a manner such that they do no dissociate from that cell during conditions experienced by the cell under methods of the invention, e.g., as described in greater detail below. Stable association is, in some instances, provided by a specific binding interaction, e.g., as described in greater detail below. In combinatorially barcoded cells of embodiments of the invention, the unique combination of sub-barcodes is stably associated with the cells using a combinatorial protocol that associates a unique combination of specific binding member/oligonucleotide sub-barcodes with a given cell, where the unique combination is obtained from an initial collection of specific binding member/oligonucleotide sub-barcodes. Combinatorial protocols employed in embodiments of the invention include split/pool protocols, e.g., as described in greater detail below.

Sub-barcodes that collectively provide a unique combinatorial barcode to a cell are provided by specific binding member/oligonucleotide sub-barcodes. Specific binding member/oligonucleotide sub-barcodes include a specific binding member component and oligonucleotide sub-barcode component, where the specific binding member component and oligonucleotide sub-barcode component are stably associated with each other, e.g., by a suitable bond or linking group, e.g., covalent bond. As such, specific binding member/oligonucleotide sub-barcodes may be viewed as having a specific binding member conjugated to an oligonucleotide sub-barcode component. Embodiments of each of these components is now described in greater detail.

The specific binding member components of specific binding member/oligonucleotide sub-barcodes employed in embodiments of the invention may vary. The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. Specific binding members may vary, where examples of specific binding members include, but are not limited to, polypeptides, nucleic acids, carbohydrates, lipids, peptoids, etc. In some instances, the specific binding member is proteinaceous. As used herein, the term “proteinaceous” refers to a moiety that is composed of amino acid residues. A proteinaceous moiety can be a polypeptide. In certain cases, the proteinaceous specific binding member is an antibody. In certain embodiments, the proteinaceous specific binding member is an antibody fragment, e.g. a binding fragment of an antibody that specific binds to a polymeric dye. As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (κ), lambda (I), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The term antibody is meant to include full length antibodies and may refer to a natural antibody from any organism, an engineered antibody, or an antibody generated recombinantly for experimental, therapeutic, or other purposes as further defined below. Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions. In certain embodiments, the specific binding member is a Fab fragment, a F(ab′)₂ fragment, a scFv, a diabody or a triabody. In certain embodiments, the specific binding member is an antibody. In some cases, the specific binding member is a murine antibody or binding fragment thereof. In certain instances, the specific binding member is a recombinant antibody or binding fragment thereof.

The specific binding member/oligonucleotide sub-barcodes may specifically bind to any convenient cell marker. In some instances, the specific binding member/oligonucleotide sub-barcodes bind to cell surface markers, where cell surface markers of interest include, but are not limited to, ubiquitous cell surface markers, i.e., cell surface markers that are at least predicted to be on all cells of a given cellular sample to be processed in a given workflow in accordance with the present invention. Examiner of ubiquitous cell surface markers to which specific binding member/oligonucleotide sub-barcodes may specific bind include, but are not limited to: CD44, CD45, P-2 microglobulin, and the like.

In addition to the specific binding member component, specific binding member/oligonucleotide sub-barcodes also include an oligonucleotide sub-barcode component. Oligonucleotide sub-barcode components may vary in length, ranging in some instances from 10 to 500 nt, such as 15 to 100 nt. In some instances, the oligonucleotide sub-barcode components may be made up of ribonucleic acids or deoxyribonucleic acids, as desired. Oligonucleotide sub-barcodes of embodiments of the invention may include an image label region, as well as other domains that find use in embodiments of the invention, where such domains may include a unique identifier for the specific binding member, a capture sequence, a primer binding site, etc.

An image label region of an oligonucleotide sub-barcode component is a domain or subsequence, i.e., stretch, of the oligonucleotide sub-barcode components that services a specific binding site for a labeled oligonucleotide employed in the imaging step of embodiments of the invention, e.g., as described in great detail below. The sequence of an image label region can be employed as an identifier of a label, such a fluorescent label, of a labeled oligonucleotide that hybridizes to the image label region. As such, the sequence of an image label region corresponds to the label of the labeled oligonucleotide that binds to that image label region. The image label region may have any convenient sequence and vary in length, in some instances ranging from 5 to 100 nt, such as 10 to 50 nt. A given oligonucleotide sub-barcode component may include a single image label region, or two or more image label regions, such as three or more image label regions, where in some instances the number of image label regions ranges from one to five, such as two to three.

In addition to the image label region, the oligonucleotide sub-barcode component may include one or more of a unique identifier for the specific binding member, a capture sequence, a primer binding site and the like. A unique identifier for the specific binding member is a domain or region that may be employed, e.g., by its sequence, to identify the specific binding member. The unique identifiers can be, for example, a nucleotide sequence having any suitable length, for example, from about 4 nucleotides to about 200 nucleotides. In some embodiments, the unique identifier is a nucleotide sequence of 25 nucleotides to about 45 nucleotides in length. In some embodiments, the unique identifier can have a length that is, is about, is less than, is greater than, 4 nucleotides, 5 nucleotides, 6 nucleotides, 7 nucleotides, 8 nucleotides, 9 nucleotides, 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 70 nucleotides, 80 nucleotides, 90 nucleotides, 100 nucleotides, 200 nucleotides, or a range that is between any two of the above values.

The oligonucleotide component may include a capture sequence, e.g., which is a domain or region that serves as a binding site for target binding region, e.g., of a bead bound barcode nucleic acid, such as described above. Capture sequences of interest may vary, as desired, and may be specific or random or semi random. In some instances, the capture sequence is that hybridizes to a target binding region of a bead bound nucleic acids, e.g., as described in greater detail below. In some instances the capture sequence is a poly(A) sequence, which poly(A) sequence is configured to hybridize to an oligodT target binding region, such as described in greater detail below. In such instances, the length of the poly(A) capture sequence may vary, ranging in some instances from 3 to 50, such as 5 to 25 nt. When present, the capture sequence may be positioned at the 5′ end of the oligonucleotide component.

Oligonucleotide components may include a primer binding site. A primer binding site, when present, may be configured to bind to a primer employed, e.g., in preparing sequenceable nucleic acids. For example, an oligonucleotide component may include a universal primer. A universal primer can refer to a nucleotide sequence that is universal or common across all specific binding member/oligonucleotide sub-barcodes employed in a given workflow. In some instances, a primer binding site can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these nucleotides in length. A primer binding site can vary in length, and can be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 nucleotides in length. A universal primer can vary in length, and in some instances can range from 5-30 nucleotides in length. The primer binding site can be positioned at the 5′ end of the oligonucleotide sub-barcode component.

As mentioned above, in specific binding member/oligonucleotide sub-barcodes, a specific binding member is conjugated to an oligonucleotide sub-barcode component. The oligonucleotide component can be conjugated with the specific binding member component through various mechanisms. In some embodiments, the oligonucleotide component can be conjugated with the specific binding member component covalently. In some embodiments, the oligonucleotide component can be conjugated with the specific binding member component non-covalently. In some embodiments, the oligonucleotide component is conjugated with the specific binding member component reagent through a linker. The linker can be, for example, cleavable or detachable from the specific binding member and/or oligonucleotide components. In some embodiments, the linker can include a chemical group that reversibly attaches the oligonucleotide to the specific binding member. The chemical group can be conjugated to the linker, for example, through an amine group. In some embodiments, the linker can comprise a chemical group that forms a stable bond with another chemical group conjugated to the specific binding member component. For example, the chemical group can be a UV photocleavable group, a disulfide bond, a streptavidin, a biotin, an amine, etc. In some embodiments, the chemical group can be conjugated to the specific binding member component through a primary amine on an amino acid, such as lysine, or the N-terminus. Commercially available conjugation kits, such as the Protein-Oligo Conjugation Kit (Solulink, Inc., San Diego, California), the Thunder-Link® oligo conjugation system (Innova Biosciences, Cambridge, United Kingdom), etc., can be used to conjugate the oligonucleotide component to the specific binding member component. The oligonucleotide component can be conjugated to any suitable site of the specific binding member component (e.g., a protein binding reagent), as long as it does not interfere with the specific binding between the specific binding member component and its cellular component target. Methods of conjugating oligonucleotides to specific binding members (e.g., antibodies) have been previously disclosed, for example, in U.S. Pat. No. 6,531,283, the contents of which are incorporated herein by reference. Stoichiometry of oligonucleotide to specific binding member can be varied.

Further details regarding specific binding member/oligonucleotide sub-barcode reagents and components thereof that find use in embodiments of the invention are provided in U.S. Patent Application Publication No. US2018/0088112; US Patent Application Publication No. 2018/0200710; U.S. Patent Application Publication No. US2018/0346970; U.S. Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference.

A given combinatorially barcoded cell may include one or more specific binding member/oligonucleotide sub-barcodes stably associated therewith. In some instances, a given combinatorially barcoded cell includes a plurality, i.e., two or more, distinct specific binding member/oligonucleotide sub-barcodes stably associated therewith, where the different specific binding member/oligonucleotide sub-barcodes differ from each other at least with respect to the cell marker, e.g., cell surface protein, to which they specifically bind. In some instances the number of distinct specific binding member/oligonucleotide sub-barcodes stably associated with a combinatorially labeled cell ranges from two to ten, such as two to five, e.g., three to four.

In embodiments of methods of the invention, cells of cellular samples may be combinatorially barcoded using any convenient protocol. In some instances, combinatorially barcoding comprises one or more split/pool iterations that sequentially contacts cells of the cellular sample with different specific binding member/oligonucleotide sub-barcodes. In some instances, each split/pool iteration comprises: apportioning cells of the cellular sample into different compartments; introducing different (i.e., distinct) specific binding member/oligonucleotide sub-barcodes that differ from each other by oligonucleotide sub-barcode component into the different compartments to produce sub-barcoded cells; and pooling the sub-barcoded cells of the different compartments.

In a given split/pool iteration, cells of a cellular sample are apportioned into different compartments, such that they are partitioned from each other. The number of different compartments into which the cells are apportioned may vary, and in some instances range from 5 to 1,000, such as 5 to 500 including 5 to 100, e.g., 25 to 100. In some instances, the compartments present in a substrate, such as where the compartments are wells of a well-plate, such as wells of a macro-well plate. Examples of well plates into which a cellular sample may be apportioned include 36 well plates, 96 well plates and 384 well plates, where in some embodiments the well plate is a 36 or 96 well plate. To apportion the cells of the cell sample into different compartments, any convenient protocol may be employed, e.g., dispensing, such as pipetting, aliquots of the cellular sample into the compartments, flowing sample over the surface of the well plate, etc.

Following apportionment of the cells, different specific binding member/oligonucleotide sub-barcodes that differ from each other by oligonucleotide sub-barcode component are introduced into the different compartments to produce sub-barcoded cells. A different specific binding member/oligonucleotide sub-barcode may be introduced into each compartment, such that cells of different compartments are stably associated with specific binding member/oligonucleotide sub-barcodes introduced into those compartments. In this manner, cells of different compartments are stably associated with different specific binding member/oligonucleotide sub-barcodes. In this step, the number of different specific binding member/oligonucleotide sub-barcodes that is introduced into different compartments may vary, ranging in some instances from 5 to 1,000, such as 5 to 500, where in some instances the number approximates the number of compartments. Compartmentalized cells that are stably associated with a specific binding member/oligonucleotide sub-barcode may be referred to as sub-barcoded cells.

Following production of sub-barcoded cells, the sub-barcoded cells of the different compartments may be combined or pooled, e.g., to produce a pooled composition of sub-barcoded cells. The sub-barcoded cells may be combined or pooled using any convenient protocol. For example, the liquid compositions of the different compartments made be retrieved from the compartments and combined, e.g., into a suitable tube of sufficient volume.

Each split/sub-barcode/pool sequence in a given combinatorial labeling workflow may be referred to as an iteration. A given combinatorial labeling workflow may have any desired number of iterations, with more iterations providing for more complex barcodes and there larger number of cells that may be processed in a given assay. In some instances, the number of split/pool iterations ranges from two to ten, such as two to five.

### Partitioning Combinatorial Barcoded Cells to Produce Partitioned Combinatorial Barcoded Single Cells Each Having a Combinatorial Barcode

Following production of the combinatorial barcoded cells, e.g., as described, embodiments of the methods include partitioning the combinatorial barcoded cells to produce partitioned combinatorial barcoded single cells each having a combinatorial barcode. In some instances, the partitioning includes distributing the combinatorial barcoded cells into partitions or compartments so that compartments include single combinatorial barcoded cells. By partitioning is meant that the combinatorial barcoded cells are placed into small reaction chambers, which may be fluidically isolated structures defined by solid materials, such as microwells, configured to accommodate the combinatorial barcoded cells. In some embodiments of the disclosed methods, devices, and systems, a plurality of microwells that are randomly distributed across a substrate are used. In some embodiments, the plurality of microwells are distributed across a substrate in an ordered pattern, e.g. an ordered array. In some embodiments, a plurality of microwells are distributed across a substrate in a random pattern, e.g., a random array. The microwells can be fabricated in a variety of shapes and sizes. Appropriate well geometries include, but are not limited to, cylindrical, elliptical, cubic, conical, hemispherical, rectangular, or polyhedral, e.g., three dimensional geometries comprised of several planar faces, for example, rectangular cuboid, hexagonal columns, octagonal columns, inverted triangular pyramids, inverted square pyramids, inverted pentagonal pyramids, inverted hexagonal pyramids, or inverted truncated pyramids. In some embodiments, non-cylindrical microwells, e.g. wells having an elliptical or square footprint, may offer advantages in terms of being able to accommodate larger cells. In some embodiments, the upper and/or lower edges of the well walls may be rounded to avoid sharp corners and thereby decrease electrostatic forces that may arise at sharp edges or points due to concentration of electrostatic fields. Thus, use of rounded off corners may improve the ability to retrieve beads from the microwells. Microwell dimensions may be characterized in terms of absolute dimensions. In some instances, the average diameter of the microwells may range from about 5 μm to about 100 μm. In other embodiments, the average microwell diameter is at least 5 μm, at least 10 μm, at least 15 μm, at least 20 μm, at least 25 μm, at least 30 μm, at least 35 μm, at least 40 μm, at least 45 μm, at least 50 μm, at least 60 μm, at least 70 μm, at least 80 μm, at least 90 μm, or at least 100 μm. In yet other embodiments, the average microwell diameter is at most 100 μm, at most 90 μm, at most 80 μm, at most 70 μm, at most 60 μm, at most 50 μm, at most 45 μm, at most 40 μm, at most 35 μm, at most 30 μm, at most 25 μm, at most 20 μm, at most 15 μm, at most 10 μm, or at most 5 μm. The volumes of the microwells used in the methods of the invention may vary, ranging in some instances from about 200 μm³ to about 800,000 μm³. In some embodiments, the micro well volume is at least 200 μm³, at least 500 μm³, at least 1,000 μm³, at least 10,000 μm³, at least 25,000 μm³, at least 50,000 μm³, at least 100,000 μm³, at least 200,000 μm³, at least 300,000 μm³, at least 400,000 μm³, at least 500,000 μm³, at least 600,000 μm³, at least 700,000 μm³, or at least 800,000 μm³. In other embodiments, the microwell volume is at most 800,000 μm³, at most 700,000 μm³, at most 600,000 μm³, 500,000 μm³, at most 400,000 μm³, at most 300,000 μm³, at most 200,000 μm³, at most 100,000 μm³, at most 50,000 μm³, at most 25,000 μm³, at most 10,000 μm³, at most 1,000 μm³, at most 500 μm³, or at most 200 μm³. The number of microwells in a given device employed in embodiments of the invention may vary, where in some instances the number is 100 or more, such as 250 or more, e.g., 500 or more, including 1000 or more, such as 5,000 or more, e.g., 10,000 or more, wherein some instances the number is 15,000 or less, e.g., 12,500 or less. Microwells suitable for use in embodiments of the invention are further described in PCT application serial no. PCT/US2016/014612 published as WO/2016/118915, the disclosure of which is herein incorporated by reference. As used herein, a substrate can refer to a type of solid support. A substrate can, for example, comprise a plurality of microwells. For example, a substrate can be a well array comprising two or more microwells. In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports. In some embodiments, a microwell can entrap only one solid support. In some embodiments, a microwell entraps a single cell and a single solid support (e.g., a bead). While the number of wells, e.g., microwells, in a well plate, e.g., microwell array, may vary in a given apportioning step, in some instances the number ranged from 5 to 500, such as 5 to 100.

In partitioning combinatorial barcoded cells, the combinatorial barcoded cells may be positioned in compartments, e.g., microwells of a microwell array, using any convenient protocol. The disclosure provides for methods for compartmenting the combinatorial barcoded cells into partitions in order to partition the combinatorial barcoded cells. A collection of combinatorial barcoded cells, for example, can be introduced into structures, e.g., microwells, to partition the combinatorial barcoded cells. The combinatorial barcoded cells can be contacted, for example, by gravity flow wherein combinatorial barcoded cells can settle into the partitioning structures. In some instances, an aqueous composition of the combinatorial barcoded cells is contacted with, e.g., by flowing it across, an array of microwells such that combinatorial barcoded cells are deposited into the microwells. The aqueous composition that includes the combinatorial barcoded cells may be flowed through a flow cell in fluidic communication with the microwells. Suitable protocols and systems for partitioning the capture particles into microwells are described in Microwells suitable for use in embodiments of the invention are further described in PCT application serial no. PCT/US2016/014612 published as WO/2016/118915, the disclosure of which is herein incorporated by reference. To partition the cells of the cell sample, any convenient protocol may be employed, e.g., dispensing, such as pipetting, aliquots of the cellular sample into the compartments, flowing sample over the surface of the well plate, etc.

In some embodiments, partitioning a plurality of combinatorial barcoded cells further includes providing a particle, e.g., bead, that includes a particle, e.g., bead, bound nucleic acid into partitions that include the single cells, where the bound nucleic acid is employed in preparing nucleic acid sequence ready compositions, e.g., sequence ready libraries, from the combinatorial barcoded cells. In some instances, the particle, e.g., bead, bound nucleic acid includes a target binding region, e.g., that binds to complementary sequences in nucleic acid species of interest in the combinatorial cell as well as to capture sequences of the oligonucleotide sub-barcode components. For example, where target nucleic acids species are cellular mRNA and the oligonucleotide sub-barcode components include a poly(A) capture sequence, a bead bound nucleic acid may include a poly (T) domain as a target binding region. In addition to the target binding region, the bound nucleic acid many further include one or more additional domains, such as but not limited to: cell label domains, barcode domains, molecular index domains (e.g., unique molecular identifier (UMI) domain), universal primer binding domains, etc. Further details regarding particles having bound nucleic acids that may be provided in compartments may be found in in U.S. Patent Application Publication No. US2018/0088112; US Patent Application Publication No. 2018/0200710; U.S. Patent Application Publication No. US2018/0346970; U.S. Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference. Beads having bound nucleic acids may be provided in the compartments using any convenient protocol, including but not limited to, those described above for partitioning of cells, and further described in further described in PCT application serial no. PCT/US2016/014612 published as WO/2016/118915, the disclosure of which is herein incorporated by reference. The particles, e.g., beads, may be partitioned into the cells before or after, or in some instances in combination with, the combinatorial barcoded cells, as desired.

### Obtaining Image Data and Sequence Data for the Partitioned Combinatorial Barcoded Single Cells

As summarized above, following production of the partitioned combinatorial barcoded single cells each having a combinatorial barcode, image data and sequence data is obtained for the partitioned combinatorial barcoded single cells. In embodiments, image data for the partitioned barcoded single cells is obtained prior to obtainment of sequence data for the partitioned combinatorial barcoded single cells.

### Image Data Obtainment

The partitioned combinatorial barcoded single cells may be imaged using any convenient protocol to obtain image data of the partitioned single cells. Image data that is obtained may vary. Image data may be obtained for any combinatorial barcoded cell of interest, and is obtained from partitions containing combinatorial barcoded cells of interest. The type of image data that is obtained may vary, and may include live cell image data. Any convenient protocol may be employed to obtain image data for combinatorial barcoded cells in partitions, where examples of imaging protocols that may be employed include, but are not limited to, microscopic imaging protocols, such as phase contrast microscopy, fluorescence microscopy, quantitative phase-contrast microscopy, holotomography, BD Rhapsody System, and the like. An image may be generated by, for example, fluorescent imaging. Imaging can comprise microscopy such as bright field imaging, oblique illumination, dark field imaging, dispersion staining, phase contrast, differential interference contrast, interference reflection microscopy, fluorescence, confocal, and single plane illumination, or any combination thereof. Imaging can comprise imaging a portion of the sample (e.g., slide/array). Imaging can comprise imaging at least 10, 20, 30, 40, 50, 60, 70, 80, 90, or 100% of the partitioned cells. In some instances, imaging can be done in discrete steps (e.g., the image may not need to be contiguous). Imaging can comprise taking at least 1, 2, 3, 4, 5, 6, 7, 8, 9, or 10 or more different images. Imaging can comprise taking at most 1, 2, 3, 4, 5, 6, 7, 8, 9, or 10 or more different images. Where desired, image data may include images taken from two or more distinct imaging iterations, where each imaging iteration includes a labeling step and then imaging step. In such instances, the obtaining of image data from the partitioned cells may be viewed as a cyclic imaging step.

In embodiments of the invention, obtaining image data for the partitioned combinatorial barcoded single cells includes obtaining a partition specific fluorescent barcode for the partitions of interest and therefore the combinatorial barcoded cells present therein. As such, methods may include, for each combinatorial barcoded cell of interest, obtaining a partition specific fluorescent barcode for the partition that contains the cell, and therefore for that cell. The partition specific fluorescent barcode is a collection of fluorescent signals obtained a given partition that corresponds to the image label regions of the combinatorial barcode of the cell in that partition. In some instances, the collection of fluorescent signals making a given barcode has a specific sequence, such as a temporal sequence corresponding to when it was obtained in a given workflow and/or the location of the image label region on the oligonucleotide sub-barcode component that corresponds to a given fluorescent signal of the barcode.

In some instances, the partition specific fluorescent barcode is obtained two or more imaging iterations, such as two to twenty iterations, including two to ten iterations, where each imaging iteration includes: contacting the partitioned combinatorial barcoded single cells with one or more labeled oligonucleotides that bind to an image label region(s) of an oligonucleotide sub-barcode component of a specific binding member/oligonucleotide sub-barcode to produce labeled partitioned combinatorial barcoded single cells; and capturing images of the labeled partitioned combinatorial barcoded single cells to obtain fluorescent signals from the labels of the labeled oligonucleotides hybridized to the image label regions. In such embodiments, labeled oligonucleotides are contacted with label oligonucleotides that bind to image label regions of the oligonucleotide sub-barcode components of the combinatorial barcoded cells. Label oligonucleotides are oligonucleotides that hybridize to the image label region and include a detectable label. In label oligonucleotides employed in embodiments of the invention, the detectable label may be part of a label nucleic acid that hybridizes to the image label region of an oligonucleotide sub-barcode unit. In such instances, the label nucleic acid may vary in length, ranging in some instances from 5 to 100 nt in length, and include one or more detectable moieties bound thereto. In some embodiments, the detectable moiety includes an optical moiety, a luminescent moiety, an electrochemically active moiety, a nanoparticle, or a combination thereof. In some embodiments, the luminescent moiety comprises a chemiluminescent moiety, an electroluminescent moiety, a photoluminescent moiety, or a combination thereof. In some embodiments, the photoluminescent moiety comprises a fluorescent moiety, a phosphorescent moiety, or a combination thereof. In some embodiments, the fluorescent moiety comprises a fluorescent dye. In some embodiments, the nanoparticle comprises a quantum dot. In some embodiments, the methods comprise performing a reaction to convert the detectable moiety precursor into the detectable moiety. Detectable moieties finding use in embodiments of the invention include those described in U.S. Patent Application Publication No. US2018/0088112; US Patent Application Publication No. 2018/0200710; U.S. Patent Application Publication No. US2018/0346970; U.S. Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference.

Contacting the partitioned combinatorial barcoded single cells with one or more labeled oligonucleotides that bind to an image label region(s) of an oligonucleotide sub-barcode component of a specific binding member/oligonucleotide sub-barcode, e.g., as described above, produces labeled partitioned combinatorial barcoded single cells. In a set of labeled partitioned combinatorial barcoded single cells, the single cells in the compartment include an image label region of a sub-barcode component hybridized to a labeled oligonucleotide. To facilitate imaging, the same labeled oligonucleotide having the same label, e.g., fluorescent dye, may be contacted with all of the combinatorial labeled cells in all the partitions. Those combinatorial labeled cells having image label regions complimentary to the labeled oligonucleotide will hybridize to the labeled oligonucleotide and be detectable in a subsequent imaging step.

Following production of the labeled partitioned combinatorial barcoded single cells, the detectable label(s) of the labeled partitioned combinatorial barcoded single cells may be detected to obtain fluorescent signals from the labels. Detection of fluorescent signals may be performed using any convenient protocol, where such protocols may include excitation of the cells with light at a suitable wavelength and detection of light from labels associated with the cells. As reviewed above, signals from partitioned combinatorial barcoded cells may be obtained in successive iterations, where each detection iteration includes a labeling step and then detection step. In such instances, the obtaining of image data from the partitioned cells may be viewed as a cyclic imaging step, such that a cyclic imaging step is employed to obtain a partition specific fluorescent barcode. In such embodiments, for example, a set of partitioned combinatorial barcoded single cells may be contacted with a first labeled oligonucleotide that specifically binds to a first image domain of sub-barcode components that may be associated with different cells of the partitioned cells. A first subset of image data may be obtained from the cells. The partitioned cells may then be contacted with a second labeled oligonucleotide that specifically binds to a second image domain of sub-barcode components that may be associated with different cells of the partitioned cells, following which a second subset of image data may be obtained from the cells. This process may be repeated for any desired number of iterations. In some instances, the number of imaging iterations used to obtained image data ranges from two to twenty, such as two to ten. Between each iteration, a prior set of labeled oligonucleotides may be removed from the partitioned cells, e.g., by washing the cells. Alternatively, the label employed in a prior iteration may be inactivated so as to be undetectable in a subsequence imaging iteration. In yet other embodiments, a set of labels that is chosen for use in a given imaging iteration protocol may be selected such that the labels are distinguishable in terms of excitation and/or emission maximum. In such instances, a single labeling step may be employed where two or more different labeled oligonucleotides are introduced in partitions under hybridization conditions. Following removal of any unbound labeled oligonucleotides, the labeled partitioned cells may then be cycled through two or more imaging steps, where each imaging step differ by excitation and/or detection of the cells. As such, a partition specific fluorescent barcode for a given partition may be obtained by first contacting a given partition with multiple different labeled oligonucleotides, where a subset of the multiple different labeled oligonucleotides bind to corresponding image label regions of the specific binding member/oligonucleotide sub-barcodes associated with the cell present in the partition. Following remove of unbound labeled nucleotides, the remaining bound labeled oligonucleotides may be detected to obtain the fluorescent barcode for that partition, where the detection protocol may be iterative, such as a cyclic imaging protocol.

### Sequence Data Obtainment

Partitioning of the combinatorial barcoded cells, e.g., as described above, results in partitioned combinatorial barcoded cells that are in spatial proximity to a particle, e.g., bead, have bound cell label domain nucleic acids that include a target binding region, e.g., as described above. When cell label domain nucleic acids are in close proximity to targets of the combinatorial barcoded single cells, the targets can hybridize to the cell label domain nucleic acid. The cell label domain comprising nucleic acid can be contacted at a non-depletable ratio such that each distinct target can associate with a distinct cell label domain comprising nucleic acid having its own unique UMI, if so desired.

Following the partitioning of the combinatorial barcoded cells, as described above, the combinatorial barcoded cells can be lysed to liberate the target molecules so that the released target molecules, e.g., nucleic acids, can bind to the target binding regions of the cell label domain nucleic acids to produce captured nucleic acids. Cell lysis can be accomplished by any of a variety of means, for example, by chemical or biochemical means, by osmotic shock, or by means of thermal lysis, mechanical lysis, or optical lysis. Particles can be lysed by addition of a cell lysis buffer comprising a detergent (e.g., SDS, Li dodecyl sulfate, Triton X-100, Tween-20, or NP-40), an organic solvent (e.g., methanol or acetone), or digestive enzymes (e.g., proteinase K, pepsin, or trypsin), or any combination thereof. To increase the association of a target and a barcode, the rate of the diffusion of the target molecules can be altered by for example, reducing the temperature and/or increasing the viscosity of the lysate. In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate. In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1 M or more Tris HCl. A lysis buffer can comprise at most about 0.01, 0.05, 0.1, 0.5, or 1 M or more Tris HCL. A lysis buffer can comprise about 0.1 M Tris HCl. The pH of the lysis buffer can be at least about 1, 2, 3, 4, 5, 6, 7, 8, 9, 10, or more. The pH of the lysis buffer can be at most about 1, 2, 3, 4, 5, 6, 7, 8, 9, 10, or more. In some embodiments, the pH of the lysis buffer is about 7.5. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7%, or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7%, or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M TrisHCl, about pH 7.5, about 0.5M LiCl, about 1% lithium dodecyl sulfate, about 10 mM EDTA, and about 5 mM DTT. Lysis can be performed at a temperature of about 4, 10, 15, 20, 25, or 30° C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

Following lysis of the combinatorial barcoded cells and release of nucleic acid molecules therefrom, the nucleic acid molecules can randomly associate with the cell label domain nucleic acids of the co-localized solid support, e.g., bead. Association can comprise hybridization of a cell label domain nucleic acid's target recognition region to a complementary portion of the target nucleic acid molecule (e.g., oligo(dT) of the barcode can interact with a poly(A) tail of a target). The assay conditions used for hybridization (e.g., buffer pH, ionic strength, temperature, etc.) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo(dT), mRNA molecules can hybridize to the probes and be reverse transcribed. The oligo(dT) portion of the oligonucleotide can act as a primer for first strand synthesis of the cDNA molecule, e.g., when subject to DNA synthesis reaction conditions to produce first strand cDNA domain comprising capture nucleic acids. Cell label domain nucleic acid can also hybridize to complementary capture sequences of oligonucleotide sub-barcode components, e.g., poly(A) sequences, of the specific binding member/oligonucleotide sub-barcodes associated with the combinatorial barcoded cells. In this way, the cell label domain nucleic acids can act as primers for reverse transcription using the oligonucleotide sub-barcode as a template, e.g., as described in greater detail below.

Where desired, a given workflow may include a pooling step where a product composition, e.g., made up of captured nucleic acids, synthesized first strand cDNAs or synthesized double stranded cDNAs, is combined or pooled with product compositions obtained from one or more additional samples, e.g., combinatorial barcoded cells. In some instances, the pooling step is performed just after hybridization step between cell label domain nucleic acids and target nucleic acids, e.g., as reviewed above. The number of different product compositions produced from different samples, e.g., cells, that are combined or pooled in such embodiments may vary, where the number ranges in some instances from 2 to 1,000,000, such as 3 to 200,000, including 4 to 100,000 such as 5 to 50,000, where in some instances the number ranges from 100 to 10,000, such as 1,000 to 5,000. Prior to or after pooling, the product composition(s) can be amplified, e.g., by polymerase chain reaction (PCR), such as described in greater detail below. Once the target-cell domain label molecules have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the labeled target nucleic acid molecules from a plurality of cells.

The disclosure provides for a method to create a target-cell label domain conjugate using any convenient protocol, such as reverse transcription or nucleotide extension. The target-cell label domain conjugate can comprise the cell label domain and a complementary sequence of all or a portion of the target nucleic acid. Reverse transcription of the associated RNA molecule can occur by the addition of a reverse transcription primer along with the reverse transcriptase. The reverse transcription primer can be an oligo(dT) primer, a random hexanucleotide primer, or a target-specific oligonucleotide primer. Oligo(dT) primers can be, or can be about, 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest. Reverse transcription can occur repeatedly to produce multiple cDNA molecules. The methods disclosed herein can comprise conducting at least about 1, 2, 3, 4, 5, 6, 7, 8, 9, 10, 11, 12, 13, 14, 15, 16, 17, 18, 19, or 20 reverse transcription reactions. The method can comprise conducting at least about 25, 30, 35, 40, 45, 50, 55, 60, 65, 70, 75, 80, 85, 90, 95, or 100 reverse transcription reactions.

One or more nucleic acid amplification reactions can be performed to create multiple copies of the target nucleic acid molecules. Amplification can be performed in a multiplexed manner, wherein multiple target nucleic acid sequences are amplified simultaneously. The amplification reaction can be used to add sequencing adapters to the nucleic acid molecules. The amplification reactions can comprise amplifying at least a portion of a sample label, if present. The amplification reactions can comprise amplifying at least a portion of the cellular label and/or barcode sequence (e.g., a molecular label). The amplification reactions can comprise amplifying at least a portion of a sample tag, a cell label, a spatial label, a barcode sequence (e.g., a molecular label), a target nucleic acid, or a combination thereof. The amplification reactions can comprise amplifying 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 100%, or a range or a number between any two of these values, of the plurality of nucleic acids. The method can further comprise conducting one or more cDNA synthesis reactions to produce one or more cDNA copies of target-barcode molecules comprising a sample label, a cell label, a spatial label, and/or a barcode sequence (e.g., a molecular label).

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR.

Amplification of the nucleic acids can comprise non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a Qβ replicase (Qβ) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some embodiments, the amplification does not produce circularized transcripts.

In some embodiments, the methods disclosed herein further comprise conducting a polymerase chain reaction on the nucleic acid (e.g., RNA, DNA, cDNA) to produce a labeled amplicon (e.g., a stochastically labeled amplicon). The labeled amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample label, a spatial label, a cell label, and/or a barcode sequence (e.g., a molecular label). The labeled amplicon can be a single-stranded molecule. The single-stranded molecule can comprise DNA, RNA, or a combination thereof. The nucleic acids of the disclosure can comprise synthetic or altered nucleic acids. As such, methods may include producing an amplicon composition from the first strand cDNA domain comprising capture nucleic acids.

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can comprise less than 12-15 nucleotides. The one or more primers can anneal to at least a portion of the plurality of labeled targets (e.g., stochastically labeled targets). The one or more primers can anneal to the 3′ end or 5′ end of the plurality of labeled targets. The one or more primers can anneal to an internal region of the plurality of labeled targets. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled targets. The one or more primers can comprise a fixed panel of primers. The one or more primers can comprise at least one or more custom primers. The one or more primers can comprise at least one or more control primers. The one or more primers can comprise at least one or more gene-specific primers.

The one or more primers can comprise a universal primer. The universal primer can anneal to a universal primer binding site. The one or more custom primers can anneal to a first sample label, a second sample label, a spatial label, a cell label, a barcode sequence (e.g., a molecular label), a target, or any combination thereof. The one or more primers can comprise a universal primer and a custom primer. The custom primer can be designed to amplify one or more targets. The targets can comprise a subset of the total nucleic acids in one or more samples. The targets can comprise a subset of the total labeled targets in one or more samples. The one or more primers can comprise at least 96 or more custom primers. The one or more primers can comprise at least 960 or more custom primers. The one or more primers can comprise at least 9600 or more custom primers. The one or more custom primers can anneal to two or more different labeled nucleic acids. The two or more different labeled nucleic acids can correspond to one or more genes.

Any amplification scheme can be used in the methods of the present disclosure. For example, in one scheme, the first round PCR can amplify molecules attached to the bead using a gene specific primer and a primer against the universal Illumina sequencing primer 1 sequence. The second round of PCR can amplify the first PCR products using a nested gene specific primer flanked by Illumina sequencing primer 2 sequence, and a primer against the universal Illumina sequencing primer 1 sequence. The third round of PCR adds P5 and P7 and sample index to turn PCR products into an Illumina sequencing library. Sequencing using 150 bp×2 sequencing can reveal the cell label and barcode sequence (e.g., molecular label) on read 1, the gene on read 2, and the sample index on index 1 read.

In some embodiments, nucleic acids can be removed from the substrate using chemical cleavage. For example, a chemical group or a modified base present in a nucleic acid can be used to facilitate its removal from a solid support. For example, an enzyme can be used to remove a nucleic acid from a substrate. For example, a nucleic acid can be removed from a substrate through a restriction endonuclease digestion. For example, treatment of a nucleic acid containing a dUTP or ddUTP with uracil-d-glycosylase (UDG) can be used to remove a nucleic acid from a substrate. For example, a nucleic acid can be removed from a substrate using an enzyme that performs nucleotide excision, such as a base excision repair enzyme, such as an apurinic/apyrimidinic (AP) endonuclease. In some embodiments, a nucleic acid can be removed from a substrate using a photocleavable group and light. In some embodiments, a cleavable linker can be used to remove a nucleic acid from the substrate. For example, the cleavable linker can comprise at least one of biotin/avidin, biotin/streptavidin, biotin/neutravidin, Ig-protein A, a photo-labile linker, acid or base labile linker group, or an aptamer.

In some embodiments, amplification can be performed on the substrate, for example, with bridge amplification. cDNAs can be homopolymer tailed in order to generate a compatible end for bridge amplification using oligo(dT) probes on the substrate. In bridge amplification, the primer that is complementary to the 3′ end of the template nucleic acid can be the first primer of each pair that is covalently attached to the solid particle. When a sample containing the template nucleic acid is contacted with the particle and a single thermal cycle is performed, the template molecule can be annealed to the first primer and the first primer is elongated in the forward direction by addition of nucleotides to form a duplex molecule consisting of the template molecule and a newly formed DNA strand that is complementary to the template. In the heating step of the next cycle, the duplex molecule can be denatured, releasing the template molecule from the particle and leaving the complementary DNA strand attached to the particle through the first primer. In the annealing stage of the annealing and elongation step that follows, the complementary strand can hybridize to the second primer, which is complementary to a segment of the complementary strand at a location removed from the first primer. This hybridization can cause the complementary strand to form a bridge between the first and second primers secured to the first primer by a covalent bond and to the second primer by hybridization. In the elongation stage, the second primer can be elongated in the reverse direction by the addition of nucleotides in the same reaction mixture, thereby converting the bridge to a double-stranded bridge. The next cycle then begins, and the double-stranded bridge can be denatured to yield two single-stranded nucleic acid molecules, each having one end attached to the particle surface via the first and second primers, respectively, with the other end of each unattached. In the annealing and elongation step of this second cycle, each strand can hybridize to a further complementary primer, previously unused, on the same particle, to form new single-strand bridges. The two previously unused primers that are now hybridized elongate to convert the two new bridges to double-strand bridges. The amplification reactions can comprise amplifying at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the plurality of nucleic acids.

Amplification of the labeled nucleic acids can comprise PCR-based methods or non-PCR based methods. Amplification of the labeled nucleic acids can comprise exponential amplification of the labeled nucleic acids. Amplification of the labeled nucleic acids can comprise linear amplification of the labeled nucleic acids. Amplification can be performed by polymerase chain reaction (PCR). PCR can refer to a reaction for the in vitro amplification of specific DNA sequences by the simultaneous primer extension of complementary strands of DNA. PCR can encompass derivative forms of the reaction, including but not limited to, RT-PCR, real-time PCR, nested PCR, quantitative PCR, multiplexed PCR, digital PCR, suppression PCR, semi-suppressive PCR and assembly PCR.

In some embodiments, amplification of the labeled nucleic acids comprises non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), a Qβ replicase (Qβ), use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and/or ramification extension amplification (RAM).

In some embodiments, the methods disclosed herein further comprise conducting a nested polymerase chain reaction on the amplified amplicon (e.g., target). The amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample tag or molecular identifier label. Alternatively, the amplicon can be a single-stranded molecule. The single-stranded molecule can comprise DNA, RNA, or a combination thereof. The nucleic acids of the present invention can comprise synthetic or altered nucleic acids.

In some embodiments, the method comprises repeatedly amplifying the labeled nucleic acid to produce multiple amplicons. The methods disclosed herein can comprise conducting at least about 1, 2, 3, 4, 5, 6, 7, 8, 9, 10, 11, 12, 13, 14, 15, 16, 17, 18, 19, or 20 amplification reactions. Alternatively, the method comprises conducting at least about 25, 30, 35, 40, 45, 50, 55, 60, 65, 70, 75, 80, 85, 90, 95, or 100 amplification reactions.

Amplification can further comprise adding one or more control nucleic acids to one or more samples comprising a plurality of nucleic acids. Amplification can further comprise adding one or more control nucleic acids to a plurality of nucleic acids. The control nucleic acids can comprise a control label.

Amplification can comprise use of one or more non-natural nucleotides. Non-natural nucleotides can comprise photolabile and/or triggerable nucleotides. Examples of non-natural nucleotides include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise one or more oligonucleotides. The one or more oligonucleotides can comprise at least about 7-9 nucleotides. The one or more oligonucleotides can comprise less than 12-15 nucleotides. The one or more primers can anneal to at least a portion of the plurality of labeled nucleic acids. The one or more primers can anneal to the 3′ end and/or 5′ end of the plurality of labeled nucleic acids. The one or more primers can anneal to an internal region of the plurality of labeled nucleic acids. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled nucleic acids. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. The one or more custom primers can anneal to the first sample tag, the second sample tag, the molecular identifier label, the nucleic acid or a product thereof. The one or more primers can comprise a universal primer and a custom primer. The custom primer can be designed to amplify one or more target nucleic acids. The target nucleic acids can comprise a subset of the total nucleic acids in one or more samples. In some embodiments, the primers are the probes attached to the array of the disclosure.

In some embodiments, barcoding (e.g., stochastically barcoding) the plurality of targets in the sample further comprises generating an indexed library of the barcoded targets (e.g., stochastically barcoded targets) or barcoded fragments of the targets. The barcode sequences of different barcodes (e.g., the molecular labels of different stochastic barcodes) can be different from one another. Generating an indexed library of the barcoded targets includes generating a plurality of indexed polynucleotides from the plurality of targets in the sample. For example, for an indexed library of the barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Barcoding (e.g., stochastic barcoding) can include using nucleic acid barcodes or tags to label individual nucleic acid (e.g., DNA or RNA) molecules. In some embodiments, it involves adding DNA barcodes or tags to cDNA molecules as they are generated from mRNA. Nested PCR can be performed to minimize PCR amplification bias. Adapters can be added for sequencing using, for example, next generation sequencing (NGS). The sequencing results can be used to determine cell labels, molecular labels, and sequences of nucleotide fragments of the one or more copies of the targets

In certain embodiments, the methods provided further include subjecting a prepared expression library, e.g., an amplicon composition produced as described above, to a sequencing protocol, such as an NGS protocol. The protocol may be carried out on any suitable NGS sequencing platform. NGS sequencing platforms of interest include, but are not limited to, a sequencing platform provided by Illumina® (e.g., the HiSeq™, MiSeq™ and/or NextSeq™ sequencing systems); Ion Torrent™ (e.g., the Ion PGM™ and/or Ion Proton™ sequencing systems); Pacific Biosciences (e.g., the PACBIO RS II Sequel sequencing system); Life Technologies™ (e.g., a SOLiD sequencing system); Oxford Nanopore (e.g., Minion), Roche (e.g., the 454 GS FLX+ and/or GS Junior sequencing systems); or any other sequencing platform of interest. The NGS protocol will vary depending on the particular NGS sequencing system employed. Detailed protocols for sequencing, e.g., which may include further amplification (e.g., solid-phase amplification), sequencing the amplicons, and analyzing the sequencing data are available from the manufacturer of the NGS sequencing system employed.

In some instances, the methods further include employing oligonucleotide labeled cellular component binding reagents, e.g., in applications where detection, e.g., quantitation, of one of or more cellular components, e.g., surface proteins, is desired. Oligonucleotide labeled cellular component-binding reagents employed in such embodiments include a cellular component-binding reagent, e.g., antibody or binding fragment thereof, coupled to a cellular component-binding reagent specific oligonucleotide comprising an identifier sequence for the cellular component-binding reagent that the cellular component-binding reagent specific oligonucleotide is associated therewith. In such instances, the magnetic capture bead may include a nucleic acid configured to capture, e.g., specifically bind to, a domain of the cellular component-binding reagent specific oligonucleotide. In this way, protein expression may be assayed in conjunction with gene expression, e.g., where multi-ohmic analysis is desired, e.g., combined analysis of transcriptome and proteome. In such instances, the methods may include preparing the captured sample with oligonucleotide labeled cellular component binding reagents, and then provide for capture of cellular component-binding reagent specific oligonucleotides released from the capture, partitioned cells. Further details regarding use of oligonucleotide labeled cellular component-binding reagents are found in United States Published Patent Application Nos. US20180267036 and US20200248263; the disclosures of which are herein incorporated by reference.

Further details regarding methods for obtaining sequence data from single cells, e.g., as described above, are provided in U.S. Patent Application Publication No. US2018/0088112; US Patent Application Publication No. 2018/0200710; U.S. Patent Application Publication No. US2018/0346970; U.S. Patent Application Publication No. 2019/0056415; U.S. Patent Application Publication No. US 2020/0248263; U.S. Patent Application Publication No. 2020/0299672; and U.S. Patent Application Publication No. 2021/0171940, the disclosures of which are herein incorporated by reference.

The sequence protocol generates sequence data for the combinatorial barcoded cells. This sequence data can then be readily linked to image data for the combinatorial barcoded cells, such that image data and sequence data obtained from the same combinatorial barcoded cells may be paired. In other words, a given set of image data and a given set of sequence data may be linked as being obtained from the same combinatorial barcoded cell, e.g., as described in greater detail below.

Linking Image Data and Sequence Data that Share a Common Combinatorial Barcode

Following obtainment of image data and sequence data, e.g., as described above, the obtained image and sequence data obtained from a given partition, and therefore a cell present in that partition, is linked. By linked is meant that image and sequence data are paired as originating from the same partition, and therefore combinatorial barcoded cell that was present in that partition when the image data for that partition was obtained. As such, image data and sequence data obtained from the same combinatorial barcoded cells may be paired. In other words, a given set of image data and a given set of sequence data may be identified as being obtained from the same combinatorial barcoded cell and then paired or otherwise associated with each other. In this manner, linked image and sequence data may be obtained for single cells of a cellular sample.

The image data and sequence data is linked by using the combinatorial barcode of the combinatorial barcoded cells from which the image and sequence data is obtained. In the obtained sequence data, e.g., as described above, sequence reads for both cellular targets and oligonucleotide barcode subunits of combinatorial barcoded cells are obtained. In other words, for each combinatorial barcoded cell assayed in a given work flow, the sequence of the oligonucleotide sub-barcodes associated with that cell and the sequence of target nucleic acids from that cell, e.g., mRNAs from the cell, are obtained. For each combinatorial barcoded cell, these obtained sequences are obtained using a protocol (which may be a next generation sequencing protocol), such as described above, where a library is generated from the original sequences, where each member of a given library generated from the same partition shares a common cell label. As such, sequence reads from the cellular target nucleic acids and the oligonucleotide sub-barcodes that are obtained from the same combinatorial barcoded cell all share the same cell label, i.e., they all have a common cell label. In linking the cell and image data, all reads that have the same cell label domain, i.e., that share a common cell label, from both reads of target nucleic acids and reads of oligonucleotide sub-barcodes, may be paired or linked. This pairing or linkage results in a set of reads that includes reads of both target nucleic acids and oligonucleotide sub-barcode nucleic acids, and these reads can be identified as originating from the same combinatorial barcoded cell.

Next, the resultant sequence data that includes reads of both target nucleic acids and oligonucleotide sub-barcode nucleic acids may be matched, i.e., paired or linked, with image data. As reviewed above, image data for combinatorial barcoded cells includes a series of fluorescent signals obtained from the different labeled oligonucleotides that are detected from a given combinatorial labeled cell during the imaging step. This series or collection of fluorescent signals that is obtained from the same partition may be referred to as a partition specific fluorescent barcode. Different partitions of a given workflow will have their own unique partition specific fluorescent barcode. A given fluorescent signal making up such a partition specific fluorescent barcode can be assigned to a given portion of a sequence read because the sequence of a labeled oligonucleotide from which that fluorescent signal is obtained is known. As such, each partition specific fluorescent barcode obtained for a given combinatorial barcoded cell that is present in that partition can be used to determine the sequences of the different image label regions associated with that combinatorial barcoded cell. As the sequences of the image label regions are present in the reads of the oligonucleotide sub-barcodes, a given partition specific fluorescent barcode may be determined as being associated with a given set of sequence data. Once a partition specific fluorescent barcode is associated with the given set of sequence data, the sequence data can be determined as being obtained from the same combinatorial barcoded cell that was in that partition from which the partition specific fluorescent barcode was obtained. In other words, from a series of fluorescent signals obtained from a given partition, a series of sequences of image label regions may be obtained for that given partition. This series or collection of sequences of image label regions may then be used to identify all sequence data obtained from that partition. This identification may be done by determining that sequence reads having both: (a) a common cell bar code; and (b) the partition identifying collection of sequences of image label regions; are obtained from a combinatorial cell that was present in a partition from which the partition specific fluorescent barcode was obtained. Once the sequence data is assigned to a given partition, the sequence data may then be readily linked with image data obtained from that partition. In this manner, linked image and sequence data may be obtained for single cells of a cellular sample.

### Kits

Aspects of the invention further include kits and compositions that find use in practicing various embodiments of methods of the invention. Kits of the invention may include: a population of specific binding member/oligonucleotide sub-barcodes; a population of labeled oligonucleotides that bind to image label regions of oligonucleotide sub-barcode components of the specific binding member/oligonucleotide sub-barcodes; and beads comprising a bead bound nucleic acid comprising a cell label domain and target binding region, e.g., as described above. The population of specific binding member/oligonucleotide sub-barcodes may include a varying number of distinct specific binding member/oligonucleotide sub-barcodes that differ from each other with respect to specific binding member and/or oligonucleotide sub-barcode, e.g., that differ from each other with respect to the image label region(s) present in the sub-barcode component. While the number of distinct specific binding member/oligonucleotide sub-barcodes a given population may vary, in some instances the number ranges from 5 to 1,000, such as 10 to 500. Populations of labeled oligonucleotides present in the kits may also vary, where in some instances the number of distinct labeled oligonucleotides that differ from each other with respect to their oligonucleotide sequences and/or labels ranges from 5 to 1,000, such as 10 to 500, e.g., 10 to 100.

The kits may further include one or more additional components finding use in practicing embodiments of the methods. For example, the kits may include components employed producing combinatorial barcoded cells, e.g., macro-well plates, liquid containers, e.g., tubes, etc. Furthermore, the kits may include one or more components employed in obtaining sequence data, e.g., one or more of: primers, a polymerase (e.g., a thermostable polymerase, a reverse transcriptase both with hot-start properties, or the like), dsDNAse, exonuclease, dNTPs, a metal cofactor, one or more nuclease inhibitors (e.g., an RNase inhibitor and/or a DNase inhibitor), one or more molecular crowding agents (e.g., polyethylene glycol, or the like), one or more enzyme-stabilizing components (e.g., DTT), a stimulus response polymer, or any other desired kit component(s), such as devices, e.g., as described above, solid supports, containers, cartridges, e.g., tubes, beads, plates, microfluidic chips, etc. Components of the kits may be present in separate containers, or multiple components may be present in a single container.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

The following is offered by way of illustration and not by way of limitation.

### EXPERIMENTAL

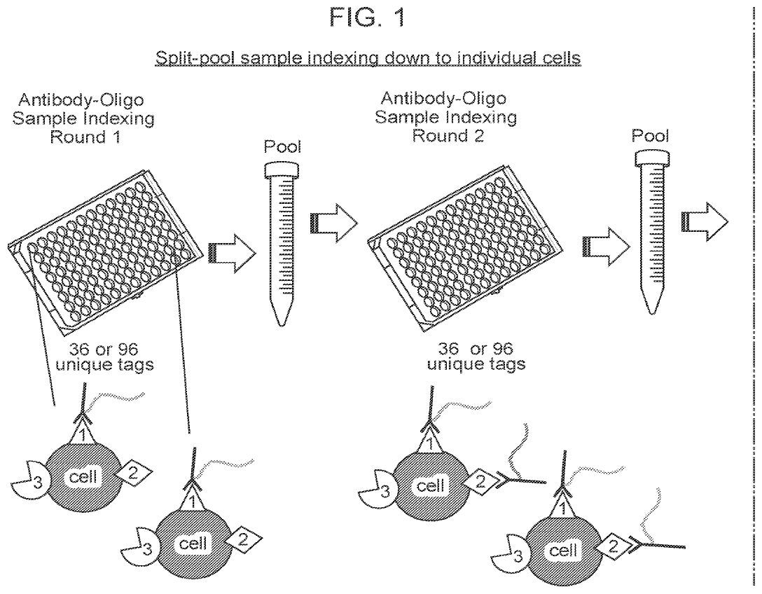

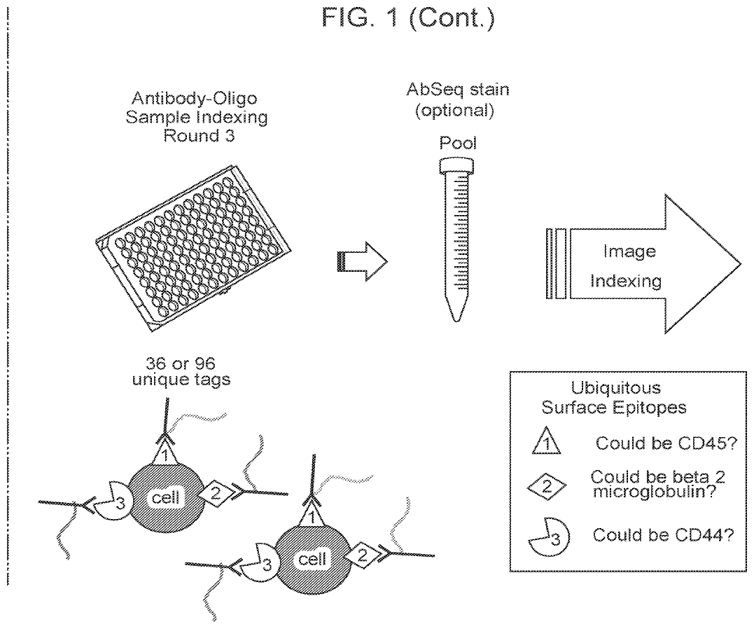

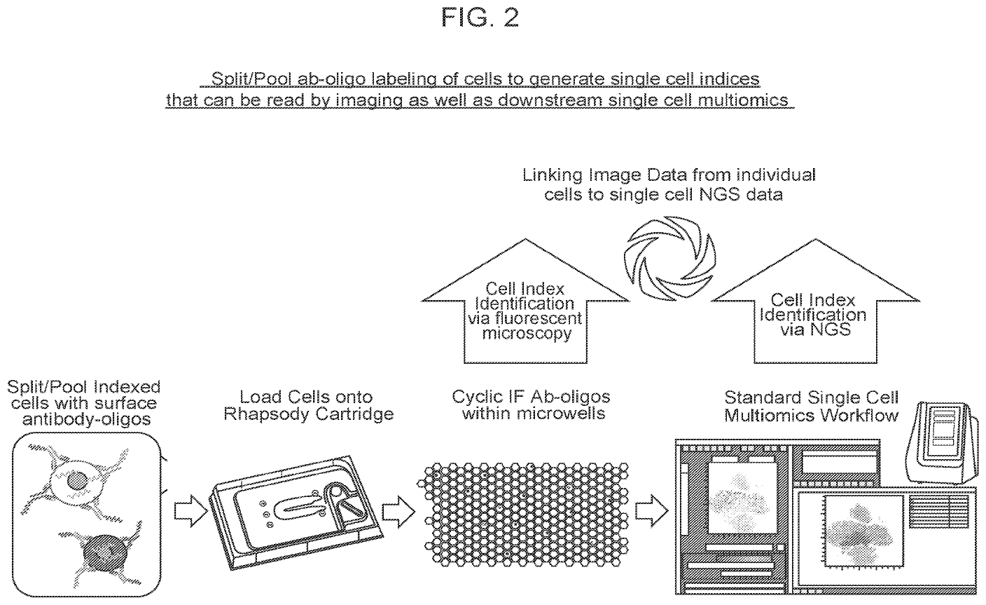

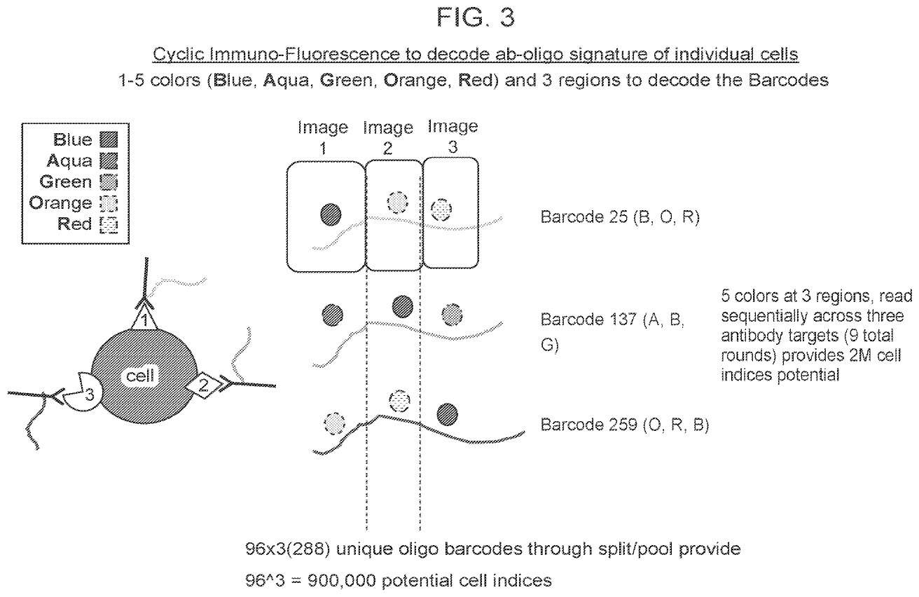

FIGS. 1 to 3 provide an illustration of a workflow in accordance with an embodiment of the invention.

Notwithstanding the appended claims, the disclosure is also defined by the following clauses:
1. A method of obtaining linked image and sequence data for single cells of a cellular sample, the method comprising:
   combinatorially barcoding cells of the cellular sample with specific binding member/oligonucleotide sub-barcodes to produce combinatorial barcoded cells;
   partitioning the combinatorial barcoded cells to produce partitioned combinatorial barcoded single cells each having a combinatorial barcode;
   obtaining image data and sequence data for the partitioned combinatorial barcoded single cells; and
   linking the image data and sequence data that share a common combinatorial barcode;
   to obtain linked image and sequence data for single cells of the cellular sample.
2. The method according to Clause 1, wherein combinatorially barcoding comprises one or more split/pool iterations that sequentially contacts cells of the cellular sample with different specific binding member/oligonucleotide sub-barcodes.
3. The method according to Clause 2, wherein each split/pool iteration comprises:
   apportioning cells of the cellular sample into different compartments;
   introducing different specific binding member/oligonucleotide sub-barcodes that differ from each other by oligonucleotide sub-barcode component into the different compartments to produce sub-barcoded cells; and
   pooling the sub-barcoded cells of the different compartments.
4. The method according to Clause 3, wherein the number of different compartments ranges from 5 to 100.
5. The method according to any of Clauses 3 and 4, wherein the compartments are wells of a well plate.
6. The method according to any of Clauses 2 to 5, wherein the number of split/pool iterations ranges from two to five.
7. The method according to any of the preceding clauses, wherein the specific binding member/oligonucleotide sub-barcodes comprise a specific binding member conjugated to an oligonucleotide sub-barcode component.
8. The method according to Clause 7, wherein the specific binding member comprises an antibody or binding fragment thereof.
9. The method according to any of Clauses 7 and 8, wherein the oligonucleotide sub-barcode component comprises an image label region.
10. The method according to Clause 9, wherein the oligonucleotide sub-barcode component further comprises one or more of a unique identifier for the specific binding member, a capture sequence and a primer binding site.
11. The method according to any of the preceding clauses, wherein the partitioning comprises distributing the combinatorial barcoded cells into partitions comprising single combinatorial barcoded cells.
12. The method according to Clause 11, wherein the distributing comprises introducing the combinatorial barcoded cells into a flow cell having microwells on a bottom surface thereof.
13. The method according to Clause 12, wherein the method further comprises providing a bead comprising a bead bound nucleic acid comprising cell label domain and a target binding region in the partitions comprising single combinatorial barcoded cells.
14. The method according to Clause 13, wherein the bead bound nucleic acid further comprises one or more of a molecular index domain and a universal primer binding domain.
15. The method according to any of the preceding clauses, wherein obtaining image data for the partitioned combinatorial barcoded single cells comprises one or more imaging iterations, each imaging iteration comprising:
   contacting the partitioned combinatorial barcoded single cells with one or more labeled oligonucleotides that bind to an image label region of an oligonucleotide sub-barcode component of a specific binding member/oligonucleotide sub-barcode to produce labeled partitioned combinatorial barcoded single cells; and
   capturing images of the labeled partitioned combinatorial barcoded single cells.
16. The method according to Clause 15, wherein the partitioned combinatorial barcoded single cells are contacted with two to five different labeled oligonucleotides that bind to different image label regions.
17. The method according to any of Clauses 15 and 16, wherein the one or more labeled oligonucleotides are fluorescently labeled.
18. The method according to any of Clauses 15 to 17, wherein the number of imaging iterations ranges from two to twenty.
19. The method according to any of the preceding clauses, wherein obtaining sequence data for the partitioned combinatorial barcoded single cells comprises employing a next generation sequencing protocol.
20. The method according to any of the preceding clauses, wherein the sequencing data comprises multiomic data.
21. A kit for obtaining linked image and sequence data for single cells of a cellular sample, the kit comprising:
   a population of specific binding member/oligonucleotide sub-barcodes;
   a population of labeled oligonucleotides that bind to image label regions of oligonucleotide sub-barcode components of the specific binding member/oligonucleotide sub-barcodes; and
   beads comprising a bead bound nucleic acid comprising a cell label domain and a target binding region.
22. The kit according to Clause 21, wherein the specific binding member/oligonucleotide sub-barcodes comprise a specific binding member conjugated to an oligonucleotide sub-barcode component.
23. The kit according to Clause 22, wherein the specific binding member comprises an antibody or binding fragment thereof.
24. The kit according to any of Clauses 22 and 23, wherein the oligonucleotide barcode component comprises an image label region.
25. The kit according to Clause 24, wherein the oligonucleotide sub-barcode component further comprises one or more of a unique identifier for the specific binding member, a capture sequence, a primer binding site
26. The kit according to any of Clauses 21 to 25, wherein the labeled oligonucleotides are fluorescently labeled.
27. The kit according to any of Clauses 21 to 26, wherein the bead bound nucleic acid further comprises one or more of a molecular index domain and a universal primer binding domain.
28. The kit according to any of Clauses 21 to 26, wherein the kit further comprises a multi-well plate.
29. The kit according to Clause 28, wherein the multi-well plate comprises a 36 to 96 well plate.
30. The kit according to any of Clauses 21 to 29, wherein the kit further comprises a flow cell having microwells on a bottom surface thereof.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of obtaining linked image and sequence data for single cells of a cellular sample, the method comprising:
combinatorially barcoding cells of the cellular sample with specific binding member/oligonucleotide sub-barcodes to produce combinatorial barcoded cells;
partitioning the combinatorial barcoded cells to produce partitioned combinatorial barcoded single cells each having a combinatorial barcode;
obtaining image data and sequence data for the partitioned combinatorial barcoded single cells; and
linking the image data and sequence data that share a common combinatorial barcode;
to obtain linked image and sequence data for single cells of the cellular sample.

2. The method according to claim 1, wherein combinatorially barcoding comprises one or more split/pool iterations that sequentially contacts cells of the cellular sample with different specific binding member/oligonucleotide sub-barcodes.

3. The method according to claim 2, wherein each split/pool iteration comprises:
apportioning cells of the cellular sample into different compartments;
introducing different specific binding member/oligonucleotide sub-barcodes that differ from each other by oligonucleotide sub-barcode component into the different compartments to produce sub-barcoded cells; and
pooling the sub-barcoded cells of the different compartments.

4. The method according to claim 3, wherein the compartments are wells of a well plate.

5. The method according to any of the preceding claims, wherein the specific binding member/oligonucleotide sub-barcodes comprise a specific binding member conjugated to an oligonucleotide sub-barcode component.

6. The method according to claim 5, wherein the specific binding member comprises an antibody or binding fragment thereof.

7. The method according to claim 6, wherein the oligonucleotide sub-barcode component comprises an image label region.

8. The method according to claim 7, wherein the oligonucleotide sub-barcode component further comprises one or more of a unique identifier for the specific binding member, a capture sequence and a primer binding site.

9. The method according to any of the preceding claims, wherein the partitioning comprises distributing the combinatorial barcoded cells into partitions comprising single combinatorial barcoded cells.

10. The method according to Clause 9, wherein the distributing comprises introducing the combinatorial barcoded cells into a flow cell having microwells on a bottom surface thereof.

11. The method according to Clause 10, wherein the method further comprises providing a bead comprising a bead bound nucleic acid comprising cell label domain and a target binding region in the partitions comprising single combinatorial barcoded cells.

12. The method according to any of the preceding claims, wherein obtaining image data for the partitioned combinatorial barcoded single cells comprises one or more imaging iterations, each imaging iteration comprising:
contacting the partitioned combinatorial barcoded single cells with one or more labeled oligonucleotides that bind to an image label region of an oligonucleotide sub-barcode component of a specific binding member/oligonucleotide sub-barcode to produce labeled partitioned combinatorial barcoded single cells; and
capturing images of the labeled partitioned combinatorial barcoded single cells.

13. The method according to any of the preceding claims, wherein obtaining sequence data for the partitioned combinatorial barcoded single cells comprises employing a next generation sequencing protocol.

14. The method according to any of the preceding claims, wherein the sequencing data comprises multiomic data.

15. A kit for obtaining linked image and sequence data for single cells of a cellular sample, the kit comprising:
a population of specific binding member/oligonucleotide sub-barcodes;
a population of labeled oligonucleotides that bind to image label regions of oligonucleotide sub-barcode components of the specific binding member/oligonucleotide sub-barcodes; and
beads comprising a bead bound nucleic acid comprising a cell label domain and a target binding region.
